(19) United States
(12) Patent Application Publication
(10) Pub. No.: US 2026/0256393 A1
(43) Pub. Date: Sep. 3, 2026

(54) TeleAutoNeuro: Autonomous Remote Head and Eye AR/VR/XR Sensing for Neurologic Screening, Diagnosis and Disease Monitoring

(71) Applicant: THE JOHNS HOPKINS UNIVERSITY, Baltimore, MD (US)

(72) Inventors: Kemar E Green, Baltimore, MD (US); Peter Kanzanzides, Lutherville, MD (US); Haochen Wei, Baltimore, MD (US); Justin C Bosley, Baltimore, MD (US)

(21) Appl. No.: 19/553,987

(22) Filed: Mar. 2, 2026

Related U.S. Application Data
(60) Provisional application No. 63/765,378, filed on Feb. 28, 2025.

(51) Int. Cl. A61B 5/16; A61B 5/00; G16H 10/20; G16H 50/20; G16H 80/00

(52) U.S. Cl. CPC A61B 5/163; A61B 5/0022; A61B 5/4023; G16H 10/20; G16H 50/20; G16H 80/00

(57) ABSTRACT

TeleAutoNeuro leverages AR/VR/XR technologies to autonomously perform real-time, quantitative assessments without the need for direct provider involvement, significantly advancing the accessibility, efficiency, and accuracy of neurologic care. TeleAutoNeuro uses augmented reality (AR), virtual reality (VR), and/or extended reality (XR) for precise head and eye movement sensing, offering a more immersive and accurate assessment environment. It provides real-time, quantitative data on neurologic function, enhancing the precision and reliability of screenings. Additionally, it significantly improves access to neurologic care for patients in remote or underserved areas and enables continuous monitoring of chronic neurologic conditions, allowing for more effective long-term disease management and timely interventions. The present invention is unique compared to other available products, because it allows for autonomy in screening and diagnosis, reducing the need for immediate specialist involvement.

### CROSS REFERENCE TO RELATED APPLICATIONS

This application claims the benefit of U.S. Provisional Patent Application No. 63/765,378 filed on Feb. 28, 2025, which is incorporated by reference, herein, in its entirety.

### FIELD OF THE INVENTION

The present invention relates generally to diagnostic tools for screening and disease monitoring. More particularly, the present invention relates to autonomous remote head and eye AR/VR/XR sensing for neurologic screening, diagnosis, and disease monitoring.

### BACKGROUND OF THE INVENTION

Dizziness/vertigo affects millions of patients in the U.S. annually. The causes of dizziness and vertigo can be neurologic or non-neurologic. The neurologic causes can be accurately delineated using key components of medical history, as well as eye movement and head position assessments. In acute settings, distinguishing rapidly and accurately between benign inner ear diseases and potentially debilitating (or fatal) neurologic injuries (usually strokes) in patients experiencing a sudden onset of constant dizziness (or AVS—acute vestibular syndrome) has garnered significant attention in neuro-otology and neuro-ophthalmology. High resolution brain imaging often fails to accurately differentiate these groups during the critical window for urgent interventions. The inadequacies of brain imaging have contributed to increased healthcare spending on unnecessary tests and increased patient disability. As a result, experts developed a three-step testing battery known as HINTS (Head Impulse, Nystagmus, and Test of Skew). This test is grounded in the physiology of the neural circuits connecting the inner ear's rotation and gravity sensors to the brain. It evaluates changes in eye movement based on gaze positions, the vertical alignment of the eyes within the orbit, and subtle eye movement reactions to high velocity/low amplitude operator-induced head rotations. In the appropriate time frame for patients with AVS, HINTS has proven to be more sensitive and specific than a brain MRI in differentiating strokes from vestibular neuritis (inner ear disease).

Although the HINTS test exists and data support its accuracy in identifying strokes in acute vertigo patients, its widespread adoption faces challenges. The primary challenges are the availability of vestibular neurology experts to interpret or administer the test and the scarcity of skilled emergency room providers trained in the technique. This dearth of skilled individuals has prompted a reimagining of the HINTS assessment, leading to the popular use of portable video oculography systems and various telemedicine solutions. Yet, none of these fully address the root problem.

While dizziness and vertigo are used here as an example, the incidence and prevalence of neurological diseases are expected to increase due to aging and the projected high incidence/prevalence of diseases like strokes, dementia, and other neurodegenerative disorders. A recent AAN survey indicates a mismatch between the available number of neurologists and the demand for tertiary care neurology services. The already long wait times for access to neurological services at tertiary care institutions are expected to worsen in the coming years. This increase will negatively impact patient outcomes and the quality of life and academic productivity of neurology faculty. The lack of general neurologists in our institution, combined with lengthy medical records and inadequate referral information, contributes significantly to current barriers to neurology access. Hence, there is a pressing need for more efficient triage methods to enable timelier and more accurate subspecialty neurology referrals.

It would therefore be advantageous to provide autonomous remote head and eye AR/VR/XR sensing for neurologic screening, diagnosis, and disease monitoring.

### SUMMARY OF THE INVENTION

The foregoing needs are met, to a great extent, by the present invention directed to a system for assessment including a computer processor configured for receiving data from a subject, wherein that data can be used for assessment of a neurological condition. The computer processor is configured for monitoring quality of the data from the subject in real time. As such, receiving data continues until a predetermined amount of data that meets a predetermined data quality standard is collected. The computer processor is also configured for transmitting the data to a health care provider.

In accordance with an aspect of the present invention, the system further includes a device configured for data collection. The device configured for data collection is one selected from a group consisting of one or more of a head mounted display-based system, a smartphone, a tablet, a personal computing device, a camera, a smartwatch, and/or a recording device. The system further includes analyzing the data to generate a diagnosis for the subject. The data received from the subject can include eye movement data. The system prompts the subject for data for an assessment. In some embodiments the assessment is a HINTS assessment. The processor is programmed to generate additional questions based on the response of the subject. The system includes transmitting the data to the subject's physician for further processing and diagnosis. The system includes transmitting the data to a data cloud for further analysis.

In accordance with an aspect of the present invention, a method includes receiving data from a subject, using a computer processing device. That data can be used for assessment of a neurological condition. The method includes monitoring, using the computer processing device, to assess quality of the data from the subject in real time. Received data continues until a predetermined amount of data that meets a predetermined data quality standard is collected. The method includes transmitting, using the computer processing device, the data for analysis.

The method includes receiving data from a device configured for data collection. The method includes analyzing the data to generate a diagnosis for the subject. The data received from the subject includes eye movement data. The method includes prompting the subject for data for an assessment. The assessment is a HINTS assessment. The processor is programmed to generate additional questions based on the response of the subject. The method includes transmitting the data to the subject's physician for further processing and diagnosis. The method includes transmitting the data to a data cloud for further analysis. The method includes generating a diagnosis for the subject.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a schematic view of TeleAutoHINTS, according to an embodiment of the present invention.

FIGS. 2A-2C illustrate schematic views of exemplary systems for patient testing and assessment according to an embodiment of the present invention.

FIG. 3 illustrates image and graphical views of skew deviation guidance in the PTI and corresponding vertical gaze position in the MPI.

FIG. 4 illustrates image and graphical views of optokinetic stimulus in the PTI and corresponding left-beating nystagmus (slow-phase to the right and fast-phase to the left) in the MPI.

FIG. 5 illustrates image and graphical views of a setup for a head-impulse test with PTI guiding head positioning and MPI showing accepted head-impulse data.

FIG. 6 illustrates HINTS testing results for the three subjects. All subjects had normal head-impulse test defined as an eye to head velocity ratio of ≥0.7.

FIGS. 7A and 7B illustrate schematic views of framework for video generation and a multimodal classifier for eye movement signatures.

FIG. 8 illustrates a schematic diagram for a wearable, rare neurological disease detecting embodiment of a system according to the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claim

TeleAutoNeuro represents a ground-breaking improvement to current technology in neurologic screening and monitoring. By leveraging AR/VR/XR technologies, it autonomously performs real-time, quantitative assessments without the need for direct provider involvement, significantly advancing the accessibility, efficiency, and accuracy of neurologic care. TeleAutoNeuro uses augmented reality (AR), virtual reality (VR), and extended reality (XR) for precise head and eye movement sensing, offering a more immersive and accurate assessment environment. It provides real-time, quantitative data on neurologic function, enhancing the precision and reliability of screenings. Additionally, it significantly improves access to neurologic care for patients in remote or underserved areas and enables continuous monitoring of chronic neurologic conditions, allowing for more effective long-term disease management and timely interventions. The present invention is unique compared to other available products, because it allows for autonomy in screening and diagnosis, reducing the need for immediate specialist involvement. One specific implementation of TeleAutoNeuro is referred to herein as TeleAutoHINTS. TeleAutoHINTS is included herein simply by way of example to illustrate how TeleAutoNeuro can be applied to a specific protocol for neurological testing. It should be noted that any other application to other protocols for testing for neurology or other assessment areas known to or conceivable to one of skill in the art are also considered to be included herein.

Annually, several million patients in the United States visit the emergency room (ER) with symptoms of vertigo or dizziness. Rapidly distinguishing between benign causes, such as inner ear disease, and more severe conditions, like strokes, necessitates performing and interpreting a three-step bedside head and eye movement assessment called HINTS (head impulse, nystagmus, and test of skew). This test is more accurate than state-of-the-art brain imaging, especially early in the disease's course when there is a limited window for safely conducting lifesaving stroke interventions. A significant barrier to its widespread adoption is the shortage of experts trained to safely perform and interpret this test in the ER. This highlights the need for automated remote assessments.

One solution to overcome this barrier is the development of a remote automated neurology screening tool powered by large language models and eye tracking technology. Neurological diseases often exhibit unique eye movement signatures that can serve as indicators of neurophysiological conditions, offering diagnostic insights. The present invention analyzes eye movement signatures along with clinical historical data to autonomously screen referrals for diseases of the central and peripheral nervous systems, making subspecialty-specific triage decisions. The present invention leverages expertise in neurology, generative AI, computer vision, natural language processing, and wearable sensing technology to implement an autonomous AR/VR remote eye sensing system, integrate large language models with electronic medical records, and curate novel digital eye movement biomarkers for brain diseases.

This AI-powered, autonomous system integrates multimodal LLMs with real-time neurophysiologic data to enhance assessment and decision-making. By leveraging eye movements, pupillometry, retinal imaging, visual field, and vestibular biomarkers, this system enables continuous physiologic evaluation, providing objective, quantitative indicators for diagnosis, lesion localization, and disease monitoring.

This system surpasses existing AI-driven tools by integrating structured and unstructured EMR data with real-time neurosensory input to refine neurology referrals, optimize subspecialty triage, and localize lesions within the nervous system, enabling early, preemptive diagnosis in select cases. The AI model will use federated learning for secure, multi-cloud data integration, with real-time adaptive feedback enabling continuous refinement and personalized diagnostics.

TeleAutoNeuro represents a novel AI-driven approach to neurologic screening, triaging, localization, and diagnosis. This scalable, autonomous digital health solution integrates neurology expertise, generative AI, computer vision, NLP, and wearable sensing to create a neurology-specific multimodal LLM. By combining real-time neurophysiologic data with longitudinal EMR records, it enhances diagnostic accuracy, precision, and efficiency.

The present invention includes an AR/VR environment. Other embodiments can include device-agnostic accessibility, enabling real-time, remote neurologic assessments. This flexibility supports integration into clinical workflows, telemedicine, and large-scale screening programs. Additionally, an open-source privacy-preserving multimodal synthetic dataset, including eye movement, pupillometric, retinal, and vestibular biomarker data, will be available to advance AI-driven neurologic diagnostics. Previous studies have shown the utility of synthetic medical data in AI model development and fine-tuning. This high-fidelity dataset will enhance model generalizability, enable rigorous validation, and accelerate AI development for neurodiagnostics across academia, clinics, and industry.

This project advances precision neurology by integrating AI-driven digital biomarkers, real-time neurosensory data, and multimodal EMR analysis. Autonomous screening, triaging, and lesion localization will streamline workflows, enable early detection, and expand access to neurology services. TeleAutoNeuro sets a new standard for AI-driven, real-time neurologic care.

TeleAutoHINTS, a specific implementation of the TeleAutoNeuro system, provides a two-part solution: (1) a head-mounted display-based head and eye tracking platform, using Microsoft Hololens 2, for automated tele-sensing and (2) an interconnected interface for real-time data visualization and analysis. TeleAutoHINTS was tested on three subjects to assess the feasibility of automated testing and evaluate the head and eye movement recordings. Preliminary results suggest that a head-mounted display-based remote self-assessment platform for acute vertigo diagnosis is technically feasible.

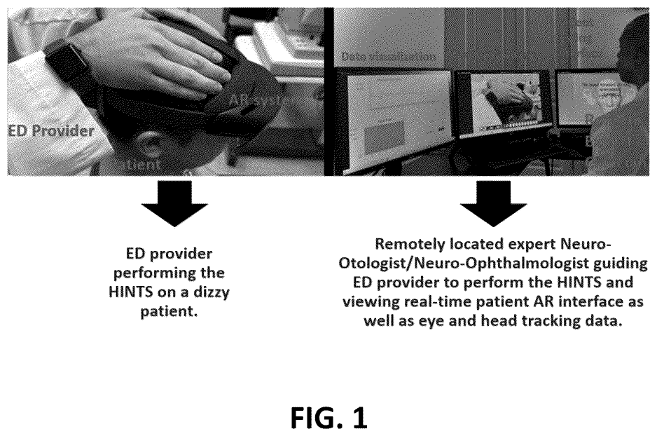

TeleAutoHINTS is a virtual/augmented reality solution for the automated tele assessment of acute vertigo patients. Preliminary patient testing was conducted to evaluate the feasibility of this innovative approach. TeleAutoHINTS, as illustrated in FIG. 1, has the potential to reduce the need for skilled operators and amplify the services of the limited expert group to health centers both nationally and internationally. The concept of remote sensing of head position and eye movement with AR and VR guidance has further clinical implications beyond assessing acute vertigo. FIG. 1 illustrates a schematic view of TeleAutoHINTS, according to an embodiment of the present invention.

Virtual and augmented reality eye tracking is an emerging area in neurology, ophthalmology, and other related fields, creating novel opportunities for research and clinical applications. In recent years, researchers have begun to compile open-source VR eye tracking datasets for various clinical and non-clinical use-cases facilitating cross-institutional AR/VR eye tracking research. In clinical neurology and ophthalmology, VR/AR eye tracking solutions have been implemented for the measurement of eye alignment D'Angelo other eye movement abnormalities, and the treatment of eye movement disorders. In vestibular neurology, VR/AR has been utilized to quantify vestibular function, evaluate balance in vestibular migraine patients, treat benign paroxysmal positional vertigo, and provide vestibular rehabilitation for various causes of dizziness, vertigo, and imbalance. To date, no one has leveraged AR or VR head-mounted displays for the automated tele-assessment of acute vertiginous patients.

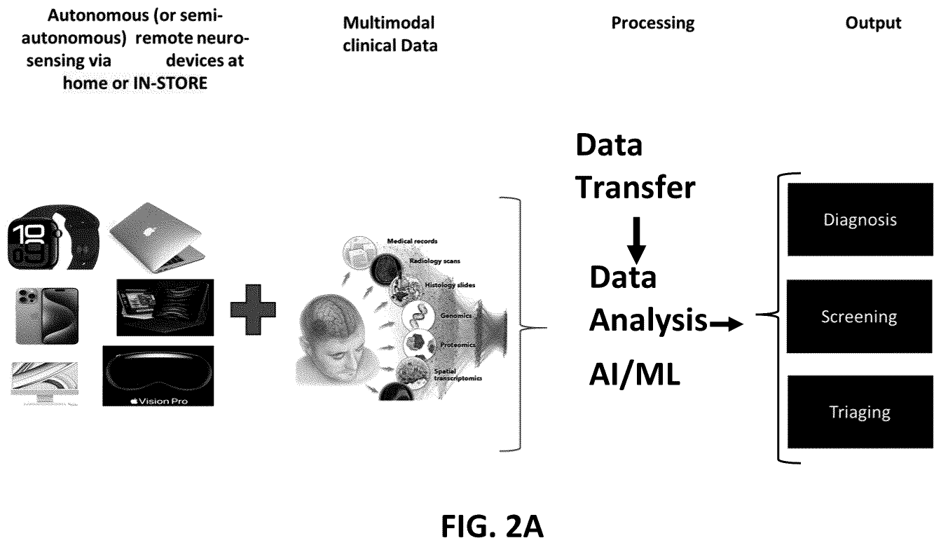

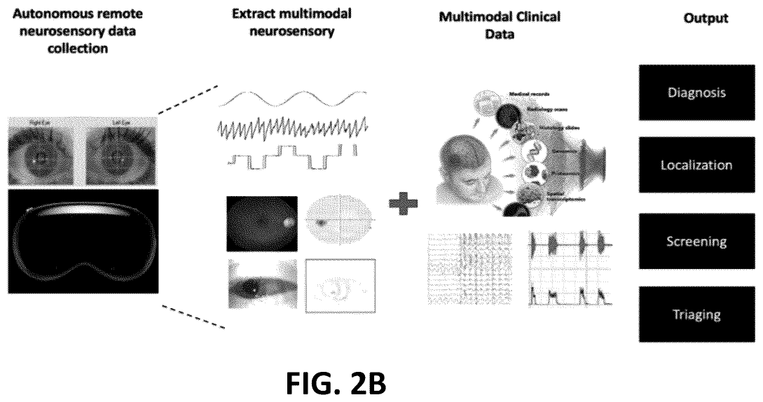

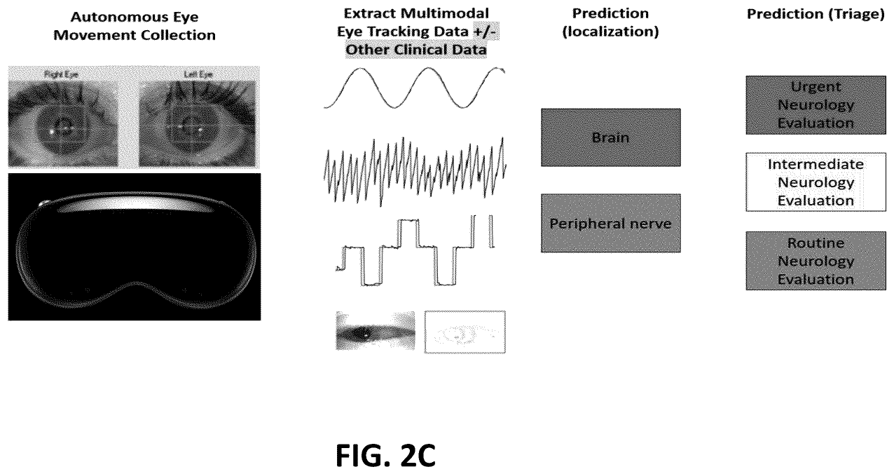

FIGS. 2A-2C illustrate schematic views of exemplary embodiments of a system for patient testing and assessment according to an embodiment of the present invention. TeleAutoNeuro leverages readily available devices to provide remote neuro-sensing. While any number of devices with sensing capabilities such as, but not limited to, LiDAR, video, gyroscopes, and accelerometers, can be used, a head mounted display-based system is used as an example herein. The head mounted display-based system is not meant to be considered limiting and the system and method of the present invention can be implemented with any hardware known to or conceivable by one of ordinary skill in the art. Data collected by the hardware can be processed to yield results about the subject's neurological condition. In some embodiments, the data collected by the hardware can be processed in conjunction with additional data and health records collected from the subject. Data can be assessed using machine learning and artificial intelligence to triage, screen, and/or diagnose the subject.

In practice, in some embodiments, the testing facilitated by the head mounted display-based system collects data, such as eye movements. The testing is led by instructions that can be automated through the head mounted display-based system or other computing device associated with the system. The instructions focus on what the subject needs to do to complete data collection. The system is configured to self-correct and ensure data integrity. As such, the system can also instruct the subject in ways to improve the quality of the data being collected. In some embodiments, testing will continue with feedback to the subject until the data is sufficient for the desired assessment.

While eye movement tracking is described herein, by way of example, other data can be collected by cameras and sensors depending on the data needed for the planned assessments. Other data types could include head motion, facial movement, gait, voice, retinal, facial expression, or other physiological data known to or conceivable by one of skill in the art.

The TeleAutoNeuro system comprises two parts: the head-mounted display-based testing platform referred to as the Patient Testing Interface (PTI), which is designed to guide patients through the test, and the desktop visualization and validation platform referring as the Medical Provider Interface (MPI). The MPI allows providers to switch between automatic data collection (patient-initiated) and quasi-automatic data collection (provider-initiated). It also offers an interface for real-time monitoring and post-test data analysis. Both interfaces were created using Unity3D (version 2021.3.28f1).

After data is collected it can be analyzed in a number of ways, as illustrated in FIGS. 2A-2C. In some embodiments the data can be transmitted to a skilled physician for analysis. In other embodiments, artificial intelligence, machine learning, and large language models can be used to analyze the data. For this analysis all data will be transformed into a readable format for the NLP systems, and patient data will be de-identified for privacy and ethical compliance. The LLM will be presented with cleaned data and queried 3 times (in a new prompt) to ensure consistency. The decisions of the models can be compared to human experts using statistical metrics like accuracy, sensitivity, specificity, and concordance.

An exemplary patient testing interface (PTI) was implemented on a Microsoft HoloLens 2, which has a sampling rate of 60 Hz and a resolution of 2048×1080 pixels. The horizontal and vertical fields of view are 430 and 290 respectively, which allows for the optimal assessment of eccentric eye gaze position data. The HoloLens' built-in eye tracker was leveraged to collect gaze data. Microsoft did not disclose their gaze tracking accuracy and precision, but Microsoft's developer website provides a rough estimate of 1.5°. The gaze data stream was obtained from each eye using the API provided by Microsoft at 60 fps. Due to Microsoft's restrictions, a consent and calibration program must be conducted before collecting gaze data. This means that all recorded gaze data has been calibrated using HoloLens 2's internal calibration program. The built-in research mode was used to obtain gyro data, which helped in calculating head position. The HoloLens did not provide any raw eye movement video data. Microsoft's Mixed Reality Toolkit (MRTK) provides a toolkit for integration between HoloLens and Unity3D (Version 2021.3.28f1). The Microsoft-provided Extended Gaze Tracking API and the HoloLens2-ResearchMode-Unity Package were used. Because HoloLens 2 is an optical see-through head-mounted display, the application of the present invention can be considered augmented reality (AR). However, in some embodiments the present invention could alternatively be implemented in virtual reality (VR), assuming the availability of sufficiently accurate gaze tracking on the platform.

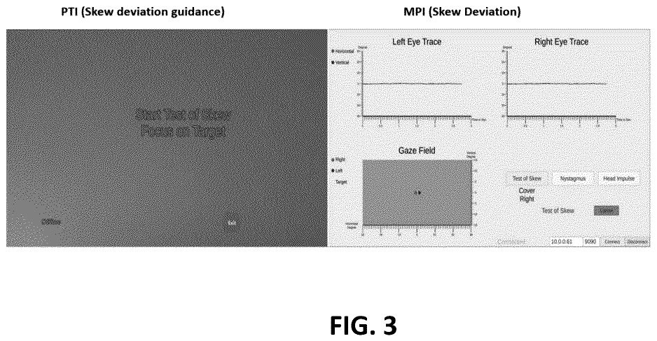

Traditionally, skew deviation is assessed by stabilizing the head and focusing on a single fixed object while alternating eye coverage. However, conducting this test solo is impractical for the patient. Thus, the cross-covering technique is simulated by displaying the target object in only one eye, eliminating the need for physical movement. Once the test starts, the PTI instructs the patient to focus on the target using displayed instructional text and voice prompts, as shown in FIG. 3. The PTI then alternates the displayed eye and records eye gaze data for test execution. The entire test duration is 15 seconds, including a 3-second initialization stage where both eyes can see the target. Data recorded during this stage is not analyzed as it is meant for patient adaptation. This is succeeded by four cross-covering stages, each 3 seconds long, where the target alternately appears in one eye. FIG. 3 illustrates image and graphical views of skew deviation guidance in the PTI and corresponding vertical gaze position in the MPI.

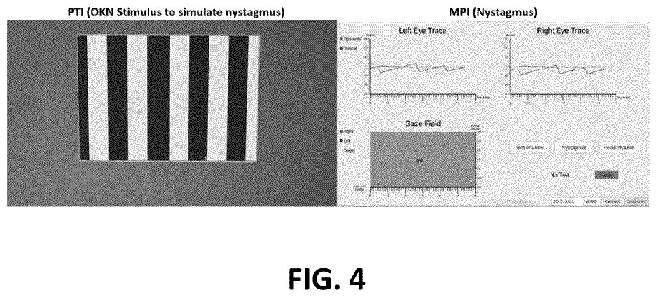

The Nystagmus test requires data collection both with and without a fixation target. The device offers text and voice guided instructions, directing patients to either fixate on a target or look away, across various eye positions. The entire test duration is 100 seconds. The first 10 seconds involve looking straight with the target displayed, followed by looking left 20 degrees for 10 seconds with the target displayed, then straight for 10 seconds, right for 10 seconds (both with the target displayed), and straight again with the target displayed for another 10 seconds. This concludes the fixation nystagmus test. The subsequent non-fixation nystagmus test sequence is: look straight for 10 seconds without target, left for 10 seconds without target, straight for 10 seconds without target, right for 10 seconds without target, and finally, straight-ahead for 10 seconds without any target. To demonstrate the system's nystagmus detection capability, physiological horizontal nystagmus was simulated using an optokinetic (OKN) stimulus moving horizontally at 1.5 Hz as shown in FIG. 4. Throughout, patients are instructed to maintain a straight-ahead gaze. The test lasts 23 seconds, but the initial 3 seconds are disregarded as patient adaptation time. FIG. 4 illustrates image and graphical views of optokinetic stimulus in the PTI and corresponding left-beating nystagmus (slow-phase to the right and fast-phase to the left) in the MPI.

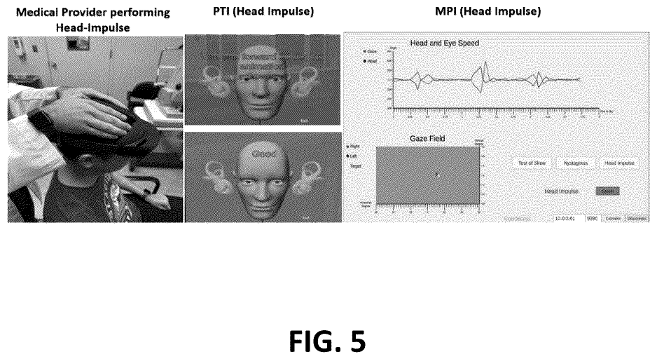

The Head Impulse test can be conducted with (passive) or without (active) the aid of an operator. Current research does not widely support active head-impulse due to potential inaccuracies and safety concerns. While anecdotal evidence suggests its utility for remote acute vertigo, more research is needed to affirm its clinical value. Therefore, a medical-provider-directed head impulse is included. In this approach, a remote medical professional instructs on-site care providers to perform passive head impulses, providing real-time feedback, as shown on the left in FIG. 5. On the PTI end, the patient is instructed to tilt their head forward by 20 degrees, ensuring they visually fixate on a target set at 0 degrees, as shown in FIG. 5. FIG. 5 illustrates image and graphical views of a setup for a head-impulse test with PTI guiding head positioning and MPI showing accepted head-impulse data. From this position, unpredictable head impulses in the yaw plane can be executed with velocities of ≥120 degrees/second and amplitudes of ≤10 degrees to either side of the visual target, allowing a latency of ≥1 second between each impulse. The target remains fixed at 0 degrees during these quick head movements. The device continuously monitors head speed and starts recording for 500 ms when the current head speed exceeds 50 degrees/sec. Head impulse results with a peak velocity of <120 degrees/sec are excluded during the data analysis phase. Data is streamed to the MPI regardless of the recording status. The device then relays the velocity to the operator in real time through the display and the MPI, ensuring optimal execution.

The Medical Provider Interface (MPI) is divided into two primary components: the User Interface and Visualization, and the Communication Portal. The User Interface and Visualization system is composed of three primary elements: Gaze Visualization (GV), Connection Control Panel (CCP), and Test Control Panel (TCP), as shown on the right in FIGS. 3-5.

The GV displays the pupil position data from both eyes for the test of skew (vertical only) and nystagmus tests (horizontal and vertical). For the head impulse test, it displays head and eye velocity-time graphs that show the head thrust and its corresponding opposite direction eye movement response. This component retains and presents gaze data from the past three seconds, enabling remote medical providers to verify the testing. It also tracks the real-time gaze field, illustrating the eye position in reference to the target position throughout the recording. For the skew deviation test, there is a text prompt that indicates in real-time which eye is covered or uncovered.

The CCP is utilized to establish and manage the connection with the PTI and to show the connection status. This is shown in the lower right corner of the MPI interface (see FIGS. 3-5).

The TCP permits medical providers to initiate, repeat, or cancel any test during the connection. Once a doctor assumes control of the test, the patient loses the ability to influence it. The test's status is also displayed within this panel, which is also in the lower right corner of the MPI interface, just above the CCP. The Communication Portal operates on a TCP socket and interfaces with the PTI. Serving as a client, it receives head and gaze data streams for visualization and validation and sends commands to oversee the testing process. There exists latency between the PTI and MPI. Although this latency was not comprehensively examined—as it can fluctuate based on the network environment—it is worth noting that, since all the data collection and testing procedures operate locally on the PTI, and all the raw data is transmitted afterward, latency is not a paramount concern for the system in the present invention.

A preliminary study was conducted to determine if the system of the present invention could collect meaningful data from patients. All participants (n=3) were adults (≥18 years old) undergoing in-person video-oculography (VOG) and video head-impulse (vHIT) testing due to complaints related to various neurological illnesses. All participants consented to participate in the research. The study received approval from the institutional review board. All subjects underwent their clinical VOG and vHIT tests before using TeleAutoHINTS, as an exemplary implementation of TeleAutoNeuro. As described above, TeleAutoHINTS comprises three subtests: skew deviation, nystagmus, and head impulse.

Post-session data analysis and visualization were performed using Matlab. The extended gaze tracking API and HoloLens 2 provided timestamps. Gaze and Head Velocity were calculated based on the eye and head position, divided by the time frame difference. All data were transmitted at 60 fps from the PTI to MPI. Data from the nystagmus test and test of skew did not undergo any post-processing. HoloLens 2 also reported the eye position in 3D space, which could be used to compensate for eye convergence. However, for testing purposes, only raw data was collected and analyzed. The Head Impulse test data was interpolated using spline interpolation built in Matlab and then plotted at 240 Hz. Inappropriate data points were identified and excluded before plotting. This includes instances like blinking during the head impulse, which leads to gaze frame loss, and non-optimal head impulses that are too slow (below 120 degrees/s).

The PTI user interface, shown on the left in FIGS. 3-5, includes several components: the visual target, an “OK” button, a “Cancel” button, instructional display text, and the connection status. Recognizing the range of technological skills among potential patients, the user interface is designed to be as simple as possible. Upon launching the program, the only visible option is an “OK” button to initiate the test. The HINTS test consists of three subtests: head impulse, nystagmus, and the test of skew, described in the following subsections. Consequently, patients must complete all subtests consecutively. Between each subtest, there is a patient-controlled resting period when the “OK” button is activated, allowing patients to proceed when ready. The “Cancel” button is available throughout the test. If pressed during a subtest, that specific subtest stops, and the ongoing test is marked incomplete. The patient can then press “OK” to restart the interrupted test. Pressing the “Cancel” button when a subtest is not active terminates the entire test session, returning the system to the phase before the first subtest. If no gaze data is detected, a warning is displayed, alerting the user to a potential calibration problem with their device. When the MPI is connected to the PTI and a test is initialized, the medical provider takes full control of the test procedure, including the ability to choose, start, or stop subtests. Once a provider takes over, patients cannot initiate test actions but can still halt or exit the PTI.

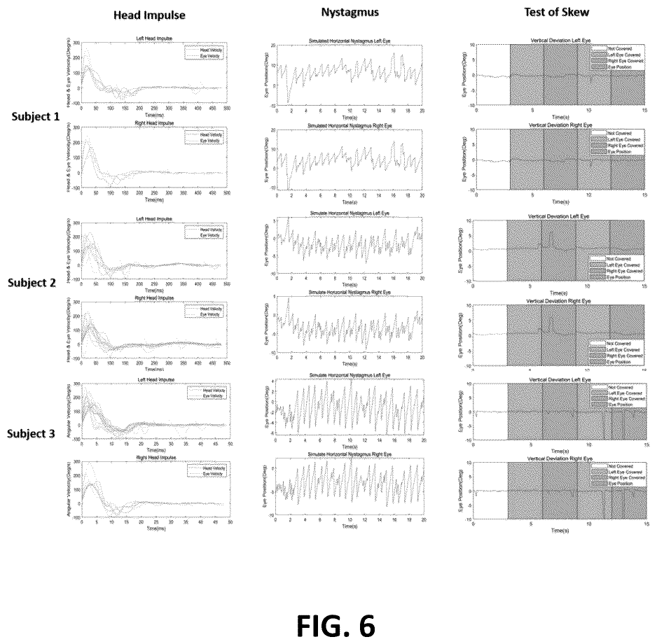

As shown in FIG. 6, the TeleAutoHINTS test results from all three subjects demonstrate that the system can produce high-quality head and eye traces that mimic physiologic eye movement responses to head impulses with the appropriate amplitude, velocity, and duration. FIG. 6 illustrates HINTS testing results for the three subjects. All subjects had normal head-impulse test defined as an eye to head velocity ratio of ≥0.7. All three nystagmus traces demonstrate fast-phases to the right (upward deflection) or right-beating nystagmus. Subject 3 had the largest amplitude of all three. All three tests of skews were normal with artifacts (blinks) in all three mimicking vertical deviation of the eyes. The head velocity data from the head impulse exceeds 120 degrees per second for all subjects, aligning with clinical standards. This is crucial since low-velocity head impulses can yield inaccurate vestibular function information. The simulated nystagmus waveforms are robust, closely resembling the typical jerk nystagmus waveform morphology with an initiating slow phase followed by a fast phase in the opposite direction. This alternating pattern of slow and fast phases (or a beat) allows medical providers to assess: 1) The direction of the nystagmus (named for the direction of the fast phase); 2) The side of the brain or inner ear affected (indicated by the direction of the slow phase), and 3) The slow-phase velocity and amplitude of the nystagmus, which can aid in differentiating between brain and inner ear nystagmus in both fixation and fixation-removed scenarios. Lastly, the test of skew measures the degree of ocular misalignment in terms of vertical eye rotation.

At its core, TeleAutoNeuro focuses on recognizing nuanced eye movement variations due to disturbances in the neural pathway, which spans from the inner ear to brainstem structures. These structures regulate eye movement and the orientation of the head with respect to the earth's vertical axis. The HINTS test profile for an acute brainstem/cerebellar stroke includes a normal head impulse test, direction-changing nystagmus, and the presence of skew deviation (vertical misalignment of the eyes). This profile is more sensitive and specific than an MRI of the brain in the first 48 hours. Despite its known limitations, the HINTS and its extended version, HINTS “plus” (which includes a hearing loss component), have consistently demonstrated their value in assessing patients with AVS in emergency settings.

Given the scarcity of VOG and HINTS-expert professionals, there is a clear demand for a tele-automated system. Regular training for emergency room staff is not always feasible. TeleAutoNeuro, and specifically TeleAutoHINTS, addresses this need by eliminating the continuous training requirement, offering a remote self-directed data collection method via a VR/AR headset. The present inventions demonstrate the feasibility of autonomously collecting high-quality eye movement data using AR/VR guidance. Initially, TeleAutoHINTS only automates the tele-sensing of certain HINTS components (nystagmus and skew test). However, its potential to reliably gather data on operator-dependent head impulses has been evident. As the system evolves, three head-impulse testing methods will be incorporated: clinician-directed (passive), robotic arm-guided (passive), and operator-independent (active). The last method will be included after its validation. Moreover, alternative techniques that avoid actual head thrusts might be integrated as proxies for head rotation to enhance telemedicine systems.

TeleAutoNeuro, and its implementation as TeleAutoHINTS, also excel in collecting quantitative data, which experts can then remotely evaluate. Recent academic efforts have explored integrating artificial intelligence (AI) into various HINTS aspects. Such progress sets the stage for future versions of the system of the present invention to leverage AI, particularly beneficial for facilities without in-house expertise. Incorporating AR/VR technologies ensures easier adoption of supplementary methods like video-ocular counter roll (vOCR), hearing tests, fundus photography, gait analysis Carmona, and others, enriching the TeleAutoHINTS process. This advantage mainly stems from AR/VR's superior immersion, adaptability, and compatibility, compared to HINTS methods based on smartphones.

Existing systems have two primary limitations. First, the absence of raw eye movement videos in the HoloLens hampers clinicians from validating ocular motor responses accurately. This feature becomes essential when eye tracking capabilities falter, necessitating direct video inspection for diagnosis. Therefore, the system of the present invention is also being deployed in a device (FOVE 0) offering raw eye tracking video outputs. The second limitation is a lack of integration with existing teleconferencing platforms. In response, the interface of the present invention is compatible with prevalent telemedicine streaming platforms.

Traditional HINTS methodologies, while clinically invaluable, face significant challenges, particularly in emergency settings due to the lack of specialized knowledge and the absence of VOG tools. It is feasible to automatically collect components of the HINTS using the AR/VR powered TeleAutoHINTS system of the present invention. This would allow remote collection of data for subjects who do not have ready access to skilled healthcare providers with this specialized knowledge. TeleAutoHINTS, harnessing the potential of AR/VR, addresses these challenges with its telemedicine-enabled self-directed protocols, modernizing essential HINTS components. As technology advances, the role of AI is set to expand, providing more streamlined analysis, especially in facilities grappling with expert shortages. By potentially integrating a wide range of diagnostic methods beyond HINTS, TeleAutoNeuro stands to revolutionize acute vestibular syndrome evaluations in emergency situations. Furthermore, this system is poised to become an invaluable tool for specialists like neuro-ophthalmologists, neuro-otologists, strabismus surgeons, and others involved in clinical roles or research within the realms of vestibular neurology and eye movement disorders. The system of the present invention allows for both the transmission of raw data to a healthcare provider for assessment and can leverage AI and machine learning algorithms to analyze the data and provide the desired assessment and interpretation of the data.

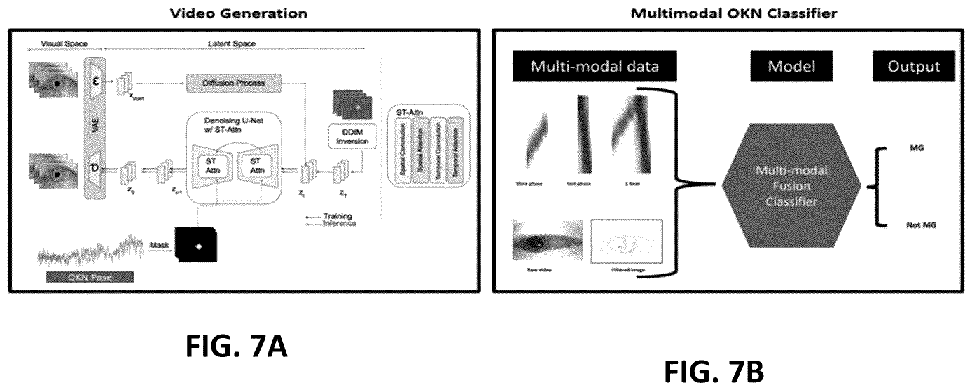

FIGS. 7A and 7B illustrate schematic views of framework for video generation and a multimodal classifier for eye movement signatures. The present invention can also be used for generating biomarkers for all diseases affecting the nervous system. One such example is eye movement signatures of myasthenia gravis (MG), a rare neurologic disease, that causes muscle fatigue with repetitive muscle use. Several eye movement types (smooth pursuit, saccades, OKN, etc.) have been implicated in MG. The present invention can be used for identifying optokinetic nystagmus (OKN) eye movement signatures. Data can be from patients and generated from known eye movement scenarios. For example, a model proficient in generating videos from segmented eye movement masks can be used. At the heart of this process is a latent video diffusion mechanism that translates segmented mask inputs into visual sequences.

From this information multimodal myasthenia gravis deep learning classifiers using imaging and waveform data can be identified. This process can be repeated for other neurologic disease states. Multimodal data (raw videos, filtered images and extracted OKN time series data) can be used to build a fusion model. The approach is based on long short-term memory (LSTM) network that is capable of processing video clips and time-series data representing OKN. The multi-modal representation is generated as a sequence of uni-modal representations (or tokens), such that the fusion module aggregates these representations through the recurrence mechanism of LSTM. The use of multimodal data in will allow for better understanding of the ocular motor biomarkers of OKN from videos and waveforms. The data being extracted from the OKN fatigue tasks (video, filtered image, waveform, etc.,) have different signatures for MG that individually might not provide the most accurate diagnosis; therefore, the combination of different data types using the multimodal deep learning approach will allow for the development of the most robust model. Furthermore, the multimodal data approach addresses the potential noise that can occur in the eye movement data, as well as the variability of the eye movement signs in MG. The models can then validated on the remaining 95-99% of the real MG data as well as a commensurate value of normal patients to assess the generalizability of the synthetic model. Various explainable AI methods will be applied to assess the model's prediction and unveil any novel “non”-physiologic.

In another proposed implementation of the present invention can be used to transform neurologic and cerebrovascular disease screening, triaging, and diagnosis through AI-driven automation. By integrating real-time neurosensing, multimodal biomarkers, and a neurology-specific LLM, the system enhances early detection, risk stratification, and stroke triage while addressing specialist shortages and inefficient referrals. Beyond neurology, this approach lays the groundwork for cardiovascular applications by leveraging retinal imaging and pupillometry, which provide insights into vascular health, autonomic function, and early markers of cardiac disease. AI-driven analysis of these biomarkers could support early detection of microvascular dysfunction and cardiometabolic risk, paving the way for AI-powered cardiac diagnostics and precision medicine at scale.

The present invention can be used as an AI-powered neurology assessment system by integrating multimodal LLMs, real-time neurophysiologic data, and synthetic datasets to enhance screening, triaging, and diagnosis across neurodegenerative, neuroinflammatory, cerebrovascular, neuromuscular, vestibular, and other neurologic conditions. AI-driven diagnostics combine clinical, imaging, neurophysiologic, and synthetic data, leveraging both existing institutional datasets and prospectively collected neuro-ophthalmic and vestibular data to improve diversity and real-world applicability.

| Data Type | Description |
|---|---|
| EMR | Clinical notes, laboratory results, and vital signs. |
| Neuroimaging | MRI, PET/CT, CT images and text reports. |
| Neurophysiologic | EEG, EMG and PSG waveforms |
| Retinal Imaging | OCT and fundus photos. |
| Eye Tracking | Video-oculography |
| Visual Fields | Humphrey visual field images |
| Pupillometry | Videos and waveforms analyzing pupil reactivity |
| Vestibular | Video-oculography. |
| Synthetic | Multimodal AI-generated twins |

The AI-driven neurosensory platform will feature multi-language support for broad accessibility. Patients can complete assessments in multiple languages with AI-driven translation and voice interaction, improving accessibility and compliance. This embodiment uses the TeleAutoNeuro wearable AR/VR neurosensing system for autonomous multimodal neuro-ophthalmic and vestibular biomarker capture.

The initial system will be developed using a FOVE 0 VR headset. However, any headset known to or conceivable by one of skill in the art can also be used. The system uses a hybrid model-based and deep learning approach for precise, real-time pupil tracking in eye tracking, perimetry (visual field), pupillometry, and semicircular vestibular assessment. Iris tracking refines otolithic function by extracting responses from subtle eye movements induced by head tilts. The system uses built-in gyroscopes for 3D head and eye tracking. The platform uses a deep-learning AI framework for real-time fundus imaging, applying neural enhancement and denoising to optimize retinal assessments on consumer-grade mobile devices. User interfaces use reinforcement learning-based adaptive UI optimization, incorporating dynamic visual guidance for neurosensory assessments, voice-assisted instructions, and real-time AI feedback to enhance patient interaction and data quality.

The neurosensing platform of the present invention can be validated by comparing FOVE 0 VR results to gold-standard clinical devices (eye tracker, fundus camera, Humphrey perimetry, pupillometry) in 10-15 healthy subjects and age-matched patients. Bland-Altman plots will assess inter-device agreement, reproducibility, and sensitivity in detecting disease-specific ocular and vestibular biomarkers. We will then deploy the validated VR platform on other commercial VR and AR headsets to evaluate cross-device performance and scalability, ensuring robust digital biomarkers for neurologic disease screening and monitoring.

Large-scale multimodal datasets that integrate real-world clinical data, prospectively collected physiologic data, and synthetic data generation can also be built using the present invention to improve AI model generalizability. By combining structured EMR data, imaging, and neurophysiologic signals, this effort will create a comprehensive dataset for AI-driven disease classification and biomarker discovery.

An autonomous EMR extraction and de-identification system functions as a real-time AI-assisted patient recruitment pipeline, leveraging contrastive self-supervised learning (SSL) frameworks, reinforcement learning-based feature selection, and multimodal transformer models to harmonize neurologic disease data while ensuring data privacy and security. This pipeline will extract retrospective multimodal clinical data from ~2,000 confirmed neurologic cases, with a 1:5 case-to-control ratio, yielding ~10,000 matched controls. To ensure regulatory compliance, all data will undergo automated encryption, de-identification, tokenization, differential privacy, and homomorphic encryption, preserving confidentiality while maintaining AI training utility. Generative AI-based tools will standardize records, mitigate missing data, and refine patient phenotyping, incorporating ICD-10 for uniformity. NLP and text-mining will extract diagnoses lacking or using outdated codes, dynamically expanding datasets. AI-driven NLP will further harmonize data by structuring insights from unstructured records while maintaining strict privacy protocols. Clinical experts will validate extracted data for specific conditions.

Neuro-ophthalmic and vestibular biomarker data can be collected, ensuring representation across targeted neurologic diseases. Each category will have ≥25 datapoints for robust AI training. Using clinical-grade devices, we will capture ocular and vestibular biomarkers to establish high-fidelity digital signatures. Existing control data will ensure proper benchmarking. Data collection will follow an IRB-approved protocol, streamlining recruitment and compliance.

To address data scarcity and privacy concerns, realistic synthetic datasets can be generated to augment clinical data and enhance AI model generalizability. Eye movement videos can be synthesized using a pose-guided diffusion model, as illustrated in FIG. 7A, which will extend to other neuro-ophthalmic and vestibular biomarkers. Pose data from 1-5% of patient data and validated open-source neuro-ophthalmic and vestibular databanks will guide synthetic data generation. Using modified generative techniques, ≥2,000 synthetic digital twins per data type per disease for robust model training. Separate deep learning models will be trained for each biomarker and validated against untouched clinical data to assess robustness and generalization. This approach enables scalable, privacy-preserving AI models for real-world neurologic diagnostics.

The present invention can also include a neurology-specific LLM integrating EMR data, imaging, and biosignals to enhance diagnostic precision, triaging, and disease classification. Multimodal representation learning will align neurophysiologic signals with clinical records, improving AI interpretability and real-world applicability. A neurology-specific multimodal LLM can be tested using EMR and synthetic neuro-ophthalmic/vestibular data testing generalizability on real-world datasets. Domain adaptation, contrastive learning, and few-shot learning will improve adaptability across neurologic phenotypes. The neurology-specific LLM of the present invention utilizes JHU Research IT and PMAP, integrating HPC resources, secure storage, and privacy-preserving AI for multimodal data processing. The LLM will be trained using a hybrid approach, combining self-supervised contrastive learning, multimodal transformers, and reinforcement learning. Federated training with differential privacy and homomorphic encryption will ensure secure AI model development across institutions. This framework enables scalable, real-time AI diagnostics in the Virtual Neurology Clinic.

To ensure clinical trust and adoption, interpretable AI methods provide transparent, meaningful explanations for model predictions. Techniques like saliency mapping, SHAP values, and attention-weighted outputs will highlight key features influencing decisions, allowing clinicians to trace AI reasoning. The system will be rigorously evaluated to ensure intuitive, actionable outputs aligned with expert expectations, fostering confidence in AI-assisted diagnostics.

Model performance is validated by benchmarking LLM-generated diagnostics against expert neurologists' gold-standard diagnoses. Retrospective validation will compare AI assessments to historical diagnoses, while prospective validation will evaluate real-time cases. Accuracy, concordance, and decision efficiency will assess AI's role in enhancing clinical workflows and neurologic care.

In another exemplary implementation of a system of the present invention, the system can be configured for detecting, diagnosing, and monitoring rare neurologic diseases. Rare neurologic diseases are difficult to diagnose due to fragmented data, misdiagnoses, and a shortage of specialists, leading to delays and missed interventions. Increasing wait times for tertiary care further hinder timely diagnosis and treatment. Current in-person diagnostic approaches limit accessibility, while AI-driven digital biomarkers remain restricted by single-modality constraints. The lack of scalable, remote, and autonomous screening tools prevents real-time, multimodal AI-assisted assessments, delaying early intervention and broader adoption in rare disease management.

The system of the present invention can be implemented as an AI-powered, autonomous wearable neuro-sensing platform for rare neurologic disease digital biomarker collection. In some embodiments the system can use Meta's Project Aria AR glasses, leveraging egocentric sensing, eye tracking, spatial mapping, and scene understanding for multimodal neurophysiologic data capture in real-world settings. It should be noted that these glasses are used as an exemplary implementation, and any AR/VR glasses known to or conceivable by one of skill in the art could also be used. Expansion to other AR/VR platforms ensures scalability, enabling real-time remote assessments across diverse populations using commercial devices, removing barriers to specialist access, and accelerating diagnosis.

The brain's visual, eye movement, and vestibular pathways provide real-time physiologic data on neurologic function. Established neuro-eye signatures exist for various neurologic diseases. For example, Myasthenia Gravis (a rare neurologic disease) can be diagnosed using eye movements alone, highlighting the potential of AI-driven multimodal neuro-sensory biomarker extraction for rare disease diagnosis. The present invention will initially be used to target rare neurodegenerative diseases, pediatric epilepsy, and neurodevelopmental disorders, expanding to other rare neurologic diseases. Camera-agnostic solutions for smartphones, tablets, laptops, and desktops ensure global scalability. Multimodal data collection across physiological domains, leveraging consumer and medical-grade devices, allows for a comprehensive, scalable neurophysiologic assessment. The table below outlines key data types, collection methods, devices, and AI processing techniques:

| Data Type | Method of Collection | Devices Used | Primary AI Processing |
|---|---|---|---|
| Oculomotor & Visual | Eye tracking, pupillometry, automated | AR/VR smart glasses, | Gaze-tracking ML models |
| Function | visual field testing | smartphones, tablets, desktops | |
| Retinal Imaging | AI-powered fundus imaging, optic nerve | AR/VR smart glasses, | Adaptive computational |
| | assessment | smartphone cameras | retinal analysis |
| Gait, Balance & Fine | Motion tracking, postural sway, | Smartphones, tablets, AR/VR, | Pose estimation |
| Motor Function | kinematic analysis | desktops | algorithms |
| Speech & Cognitive | Verbal assessments, voice biomarkers | Mobile, desktop, AR/VR | AI-based speech analysis |
| Function | using NIHSS language assessment cards | microphones | |
| Sleep & Autonomic | EEG, EOG, HRV, SpO₂, multimodal sleep | Dormotech biosensors, Apple | Federated AI sleep analysis |
| Function | tracking | SensorKit, smartwatches | models |
| Electrophysiology | EEG-based neurophysiologic monitoring | Dormotech's home EEG | ML-driven EEG feature |
| | | | extraction with EpiScalp |
| Neuroimmune & | Remote biochemical monitoring | Kiffik Bioelectronics | AI-based multi-omic |
| Metabolic Biomarkers | | | biomarker profiling |
| Clinical Data | EMR records, PROs, imaging uploads | Secure medical record portals | NLP-based data |
| Integration | | | structuring and annotation |

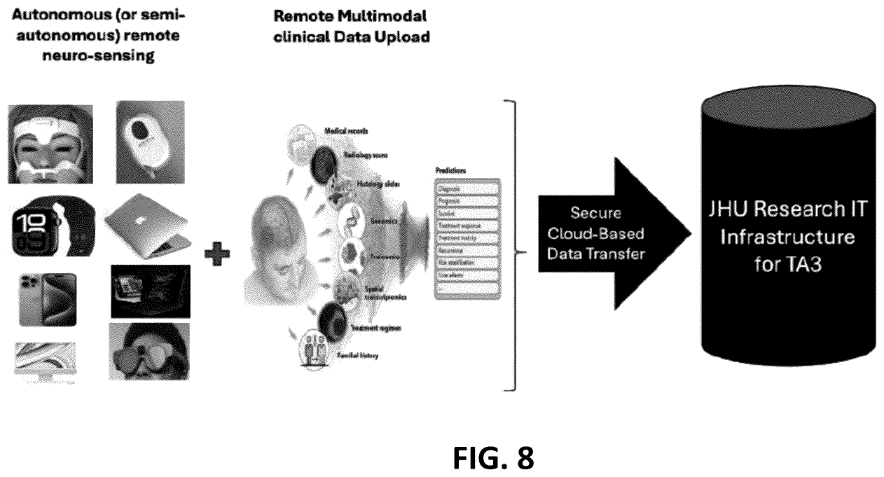

FIG. 8 illustrates a schematic diagram for a wearable, rare neurological disease detecting embodiment of a system according to the present invention. The present invention will use a scalable AI-driven neurosensory pipeline for real-time multimodal data collection, federated processing, and cross-platform deployment, as illustrated in FIG. 8. AI models process data across AR/VR, smartphones, tablets, and desktop cameras, ensuring broad accessibility and patient compliance. The system combines model-based and deep learning for real-time pupil tracking in eye tracking, pupillometry, and semicircular vestibular assessment. Iris tracking refines otolithic function by analyzing subtle eye movements from head tilts while built-in gyroscopes enable 3D head tracking and pose estimation for screen-based head-eye coordination analysis. A deep-learning AI framework will enhance fundus imaging, applying neural enhancement and denoising to optimize retinal assessments on consumer-grade mobile devices.

User interfaces leverage reinforcement learning-based optimization, integrating dynamic visual guidance, voice-assisted instructions, and real-time AI feedback to enhance patient interaction and data quality. Gait and motor function interfaces will use real-time pose estimation overlays, guiding patients through tasks with AI-driven movement segmentation. Speech interfaces feature automated NIHSS language assessment prompts, incorporate automated prompts using NIHSS language assessment cards, analyzing fluency, articulation, and recall deficits via AI-extracted acoustic biomarkers.

Additional neurosensory data from multimodal biosensing devices (Dormotech's) is supplemented by Apple SensorKit, smartwatch-derived HRV, motion, SpO₂, and biochemical data (including genomics). BevelCloud securely transfers Apple and Dormotech biosensing data to JHU IT infrastructure/InHealth PMAP and biochemical data to the JHU Precede Biomarker lab in real time. The present invention integrates AI-driven EMR extraction, automating consenting, clinical data retrieval, and real-time patient onboarding. The system structures multimodal EMR data for seamless AI integration, including diagnostics, labs, imaging, and patient-reported outcomes. Diffusion and transformer-based generative methods generate synthetic digital twins to augment datasets from all available real patient data.

The platform supports multi-language interfaces with real-time AI translation and voice interaction, enhancing accessibility and compliance. Patients can complete assessments in their preferred language, with telemedicine-based consenting and provider-assisted data collection available as needed.

This rare-disease detecting implementation of the present invention can also be expanded to all neurologic conditions, psychiatry, and neurosurgery, integrating AI-driven diagnostics into clinical care. Additional biosensors extend precision medicine into systemic disease monitoring. The federated AI platform supports autonomous screening, triaging, and diagnosis, scaling globally through industry and international partnerships.

TeleAutoNeuro addresses several significant issues in the current landscape of neurologic care:

Limited Access to Neurologic Care: Many patients, especially those in remote or underserved areas, have limited access to neurologic specialists. This invention provides a means to screen and monitor patients autonomously, thereby bridging the gap in access to care.

Provider Shortages: The healthcare industry often faces shortages of neurologists and other specialists. TeleAutoNeuro reduces the dependency on specialists for initial screenings and routine monitoring, thus alleviating some of the burden on healthcare providers.

Efficiency in Diagnosis: Traditional neurologic screening and diagnosis can be time-consuming and require multiple visits. TeleAutoNeuro streamlines the process by providing real-time, quantitative data that can inform diagnoses quickly and accurately.

Consistency and Accuracy: Human error and variability can impact the accuracy of neurologic assessments. This invention offers a standardized and objective approach to screening and monitoring, enhancing the reliability of results.

Continuous Monitoring: For chronic neurologic conditions, continuous monitoring is crucial. TeleAutoNeuro allows for regular and frequent assessments without the need for constant physical appointments, thus enabling better disease management.

Based on the capabilities described, TeleAutoNeuro could be developed into several potential products:

AR/VR/XR Headsets for Home Use: A consumer-grade headset equipped with the necessary sensors and software to allow patients to perform screenings and monitoring at home. This product would connect to a central system for data analysis and feedback.

Clinical Screening Stations: Units placed in clinics, hospitals, and healthcare facilities to provide rapid and autonomous neurologic screenings. These stations could be used for both initial patient assessments and ongoing monitoring.

Portable Screening Devices: Compact and portable devices that healthcare providers can take to remote areas or use during house calls to perform neurologic screenings and diagnostics on-site.

Integrated Health Systems: Incorporating the sensing technology into existing healthcare systems, such as electronic health records (EHRs) and telemedicine platforms, to enhance the remote monitoring and management of neurologic patients.

Mobile Application: A companion app that works with the AR/VR/XR headsets or other sensors, allowing patients to perform self-assessments and send data to their healthcare providers for review.

These products would harness the power of AR/VR/XR technology to deliver accurate, real-time data to healthcare providers, improving the overall quality and accessibility of neurologic care.

The present invention could be used globally to remotely screen and triage patients seeking tertiary neurology subspecialty care at larger health centers. This work lays the foundation for more comprehensive precision neurology screening and diagnostic tools that integrate additional multimodal clinical data, including neurophysiology, imaging, video, voice, and other kinematic data. It also paves the way for future autonomous AI screening and diagnostic tools in other non-procedural medical specialties.

The present invention can be used for testing and assessment of a number of conditions and purposes including but not limited to nystagmus, research applications, myasthenia gravis, monitoring disease states such as brain tumor, neurodegenerative diseases, clinical trials, monitoring of treatment response, monitoring and diagnosis of conditions that affect brain or vestibular pathways, concussion, sports, and military applications.

It should be noted that aspects of the system and method, its control, and calculations can be executed with a program(s) fixed on one or more non-transitory computer readable medium. The non-transitory computer readable medium can be loaded onto a computing device, microprocessor, servo, server, actuator, device processor, smartphone, tablet, phablet, a Control Box, or any other suitable device known to or conceivable by one of skill in the art. Additionally, the non-transitory computer readable medium could be incorporated into the control unit for the AR/VR device, or other control units within the assessment room. Any other suitable configuration known to or conceivable to one of skill in the art could also be employed for the execution of the present invention.

It should also be noted that herein the steps of the method described can be carried out using a computer, non-transitory computer readable medium, or alternately a computing device, microprocessor, or other computer type device independent of or incorporated with the radiation detection device. The computing device for executing the present invention can be a completely unique computer designed especially for the implementation of this method. Indeed, any suitable method of analysis known to or conceivable by one of skill in the art could be used. It should also be noted that while specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

It should be noted that the software associated with the present invention is programmed onto a non-transitory computer readable medium that can be read and executed by any of the computing devices mentioned in this application. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard disk, reel-to-reel tape, cartridge tape, cassette tapes or cards, optical media such as CD-ROM, DVD, Blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the application is transmitted either wired or wirelessly over a network, via the internet, cellular telephone network, RFID, or any other suitable data transmission means known to or conceivable by one of skill in the art.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A system for assessment comprising:
a device for data collection;
a computer processor configured for:
receiving data from a subject, wherein that data can be used for assessment of a neurological condition;
monitoring quality of the data from the subject in real time, such that receiving data continues until a predetermined amount of data that meets a predetermined data quality standard is collected; and
transmitting the data for analysis.

2. The system of claim 1 further comprising a device configured for data collection.

3. The system of claim 2 wherein the device configured for data collection comprises one or more selected from a group consisting of a head mounted display-based system, a smartphone, a tablet, a personal computing device, a camera, a smartwatch, and/or a recording device.

4. The system of claim 1 further comprising analyzing the data to generate a diagnosis for the subject.

5. The system of claim 1 wherein the data received from the subject includes eye movement data.

6. The system of claim 1 further comprising prompting the subject for data for an assessment.

7. The system of claim 6 wherein the assessment is a HINTS assessment.

8. The system of claim 6 wherein the processor is programmed to generate additional questions based on the response of the subject.

9. The system of claim 1, further comprising transmitting the data to the subject's physician for further processing and diagnosis.

10. The system of claim 1 further comprising transmitting the data to a data cloud for further analysis.

11. A method comprising:
receiving data from a subject, using a computer processing device, wherein that data can be used for assessment of a neurological condition;
monitoring, using the computer processing device, quality of the data from the subject in real time, such that received data continues until a predetermined amount of data that meets a predetermined data quality standard is collected; and
transmitting, using the computer processing device, the data for analysis.

12. The method of claim 11 further comprising receiving data from a device configured for data collection.

13. The method of claim 11 further comprising analyzing the data to generate a diagnosis for the subject.

14. The method of claim 11 wherein the data received from the subject includes eye movement data.

15. The method of claim 11 further comprising prompting the subject for data for an assessment.

16. The method of claim 15 wherein the assessment is a HINTS assessment.

17. The method of claim 15 wherein the processor is programmed to generate additional questions based on the response of the subject.

18. The method of claim 11, further comprising transmitting the data to the subject's physician for further processing and diagnosis.

19. The method of claim 11 further comprising transmitting the data to a data cloud for further analysis.

20. The method of claim 11 further comprising generating a diagnosis for the subject.
